(19) United States
(12) Patent Application Publication
(10) Pub. No.: US 2014/0315930 A1
(43) Pub. Date: Oct. 23, 2014

(54) FAST RELEASE SOLID ORAL COMPOSITIONS OF ENTECAVIR

(71) Applicants: Bandi Parthasaradhi Reddy, Hyderabad (IN); Podili Khadgapathi, Hyderabad (IN); Nelluri Ramarao, Hyderabad (IN)

(72) Inventors: Bandi Parthasaradhi Reddy, Hyderabad (IN); Podili Khadgapathi, Hyderabad (IN); Nelluri Ramarao, Hyderabad (IN)

(73) Assignee: Hetero Research Foundation, Hyderabad, Andhra Pradesh (IN)

(21) Appl. No.: 14/357,603

(22) PCT Filed: Nov. 8, 2012
(86) PCT No.: PCT/IN2012/000737

§ 371 Date: May 13, 2014

(30) Foreign Application Priority Data
Nov. 14, 2011 (IN) 3893/CHE/2011

(51) Int. Cl. A61K 31/522; A61K 9/20

(52) U.S. Cl. CPC A61K 31/522; A61K 9/2009; A61K 9/2013; A61K 9/2059; A61K 9/2054; A61K 9/205; A61K 9/2095
USPC 514/263.37

(57) ABSTRACT

The present invention is directed to fast release pharmaceutical compositions comprising entecavir or its pharmaceutically acceptable salts, process for preparing the same and use of such compositions for the treatment of Hepatitis B virus infection.

### PRIORITY

This patent application claims priority to Indian application number 3893/CHE/2011, filed on Nov. 14, 2011, the contents of which are incorporated by reference herein in their entirety.

### FIELD OF THE INVENTION

Technical field of the present invention relates to fast release solid oral compositions of entecavir or its pharmaceutically acceptable salts and process for preparing the same.

### BACKGROUND

Chemically entecavir is 2-amino-1,9-dihydro-9-[(1S,3R,4S)-4-hydroxy-3-(hydroxymethyl)-2-methylenecyclopentyl]-6H-purin-6-one, monohydrate. Its molecular formula is C₁₂H₁₅N₅O₃.H₂O, corresponding to a molecular weight of 295.3 and having the following structural formula:

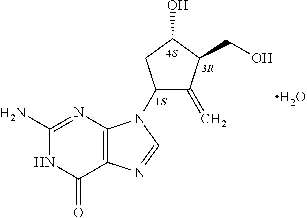

Entecavir is a guanosine nucleoside analogue indicated for the treatment of chronic Hepatitis B virus infection.

Entecavir is marketed under the trade name Baraclude® in United States by Bristol Myers Squibb in the form of oral tablets and solution.

U.S. Pat. No. 5,206,244 assigned to Squibb & Sons describes entecavir and its use as an antiviral agent.

U.S. Pat. No. 6,627,224 assigned to Bristol-Myers Squibb describes method of preparing pharmaceutical composition of entecavir by dissolving the entecavir and an adhesive substance in a solvent, followed by spraying said solution onto a carrier substrate while is in motion, then drying the coated carrier substrate to remove the solvent, and finally combining dried coated carrier substrate with other desired ingredients to form said pharmaceutical composition. The process described is time consuming, requires specialized expensive equipment like fluidized bed processor with controlled parameters such as temperature, airflow, spray rate and the like and is tedious.

Thus, there is a need to develop compositions of entecavir using simplified process that minimizes the need for specialized equipments and brings down the manufacturing cost and time.

Inventors of the present invention have developed the compositions of entecavir with specific excipients using simplified process that exhibited fast disintegration and short dissolving time with better blend/content uniformity, which were also found to be comparable with marketed Baraclude® tablets.

### SUMMARY

The present invention relates to pharmaceutical composition comprising entecavir and one or more pharmaceutically acceptable excipients and process for their preparation.

In one embodiment, the present invention relates to fast release pharmaceutical composition comprising entecavir, a diluent selected from carbonates/bicarbonates of alkali metals or alkaline earth metals and an acid component.

In another embodiment, the present invention relates to fast release pharmaceutical composition comprising entecavir, acid component, carbonates/bicarbonates of alkali metals or alkaline earth metals, superdisintegrant and one or more pharmaceutically acceptable excipients.

In another aspect, the present invention provides pharmaceutical composition comprising entecavir, acid component, carbonates/bicarbonates of sodium, magnesium, potassium and calcium, superdisintegrant and one or more pharmaceutically acceptable excipients selected from diluent(s), binder(s), lubricant(s), and glidant(s).

In another embodiment, the present invention provides wet granulation process for preparing a pharmaceutical composition comprising entecavir and at least one pharmaceutically acceptable excipient.

Accordingly, the present invention provides a process for preparing compositions of entecavir by wet granulation method involving: (i) sifting and blending one or more excipients including carbonates/bicarbonates of alkali metals or alkaline earth metals optionally with entecavir to form a dry mix, (ii) granulating the dry mix of step no. (i) using drug solution to form granules followed by drying, (iii) blending the granules of step no. (ii) with remaining portion of excipients including acid component, optionally carbonates/bicarbonates of alkali metals or alkaline earth metals and finally compressing into tablets or filled in to capsules.

Further embodiment of the present invention relates to pharmaceutical composition comprising entecavir, where in the composition is free of sweetening agents and flavouring agents.

In a preferred embodiment, the present invention relates to pharmaceutical composition comprising 0.05-1% by weight of entecavir, 1-6% by weight of acid component, 1-90% by weight of carbonates/bicarbonates of sodium, magnesium, potassium and calcium and 1-20% by weight of superdisintegrant based on total weight of the composition.

In a specific embodiment, fast release pharmaceutical tablet composition comprises entecavir, citric acid, calcium carbonate and soy polysaccharide; wherein said composition is prepared by wet granulation method.

In yet another embodiment, the pharmaceutical compositions of the present invention comprising entecavir are useful for treating chronic Hepatitis B virus infection.

### DETAILED DESCRIPTION

In accordance with the present invention the term “entecavir” includes entecavir in the form of free base, in the form of a pharmaceutically acceptable salt, amorphous entecavir, crystalline entecavir or any isomer, derivative, hydrate, solvate, or prodrug or combinations thereof.

The term “pharmaceutical composition” or “solid dosage form” or “solid oral compositions” as used herein synonymously include tablets, capsules, granules, mini-tablets and fast disintegrating tablets meant for oral administration.

The term “fast release compositions” according to the present invention refers to compositions meant for disintegration in the stomach in not more than 5 minutes, preferably less than 3 minutes, more preferably less than 1 minute.

The term “sweetening agents” refers to agents that mask the unpleasant taste of the drug.

The term “flavouring agents” refers to agents that impart flavour to the formulations.

The present invention relates to fast release pharmaceutical composition comprising entecavir, a diluent selected from carbonates/bicarbonates of alkali metals or alkaline earth metals and an acid component.

The present invention also relates to fast release pharmaceutical composition comprising entecavir, acid component, carbonates/bicarbonates of alkali metals or alkaline earth metals, superdisintegrant and one or more pharmaceutically acceptable excipients.

Suitable acid component according to the present invention include, but not limited to citric acid, tartaric acid, fumaric acid and ascorbic acid or mixtures thereof.

Suitable alkali metals according to the present invention include sodium, potassium or mixtures thereof.

Suitable alkaline earth metals according to the present invention include magnesium, calcium or mixtures thereof.

Suitable carbonates/bicarbonates of sodium, magnesium, potassium and calcium include, but not limited to sodium carbonate, magnesium carbonate, potassium carbonate, calcium carbonate, sodium bicarbonate, magnesium bicarbonate, potassium bicarbonate and calcium bicarbonate or mixtures thereof.

Suitable superdisintegrants according to the present invention include, but not limited to natural or synthetic superdisintegrants selected from soy polysaccharide, sodium starch glycolate, croscarmellose sodium, cross linked alginic acid, gellan gum and xanthan gum or mixtures thereof.

Preferably, natural superdisintegrant according to the present invention is selected from soy polysaccharide, cross linked alginic acid, gellan gum and xanthan gum or mixtures thereof. More preferably the natural superdisintegrant is soy polysaccharide.

In a preferred aspect, the present invention relates to pharmaceutical composition comprising 0.05-1% by weight of entecavir, 1-6% by weight of acid component, 1-90% by weight of carbonates/bicarbonates of sodium, magnesium, potassium and calcium and 1-20% by weight of superdisintegrant based on total weight of the composition.

More preferably, the present invention relates to pharmaceutical composition comprising 0.05-1% by weight of entecavir; 1-6% by weight of acid component; 1-90% by weight of carbonates/bicarbonates of magnesium and calcium; and 1-20% by weight of soy polysaccharide as superdisintegrant based on total weight of the composition.

More specifically, fast release pharmaceutical tablet composition comprises entecavir, citric acid, calcium carbonate and soy polysaccharide; wherein said composition is prepared by wet granulation method.

In an embodiment the present invention relates to fast release pharmaceutical composition comprising entecavir, acid component, carbonates/bicarbonates of sodium, magnesium, potassium and calcium, superdisintegrant, and one or more pharmaceutically acceptable excipients selected from diluent(s), binder(s), lubricant(s), and glidant(s).

Suitable diluents include, but are not limited to pregelatinized starch, talc, lactose, sugar, starches, modified starches, mannitol, sorbitol, inorganic salts, cellulose derivatives (e.g. microcrystalline cellulose), xylitol, lactitol, starch, kaolin, sucrose, mannitol, sorbitol, dextrates, dextrin, maltodextrin, dextrose, calcium sulfate, dibasic calcium phosphate, tribasic calcium phosphate, magnesium oxide and the like and mixtures thereof.

Suitable binders include, but are not limited to, carboxymethylcellulose sodium, pregelatinized starch, lactose, starches such as corn starch, potato starch, modified starches, sugars, guar gum, pectin, wax binders, microcrystalline cellulose, methylcellulose, carboxymethylcellulose, hydroxypropyl methylcellulose, hydroxyethyl cellulose, hydroxypropyl cellulose, copolyvidone, sodium alginate, acacia, alginic acid, tragacanth, gelatin, liquid glucose, povidone and the like and mixtures thereof.

Suitable lubricants include, but are not limited to, sodium stearyl fumarate, calcium stearate, magnesium stearate, zinc stearate, stearic acid, fumaric acid, palmitic acid, talc, carnauba wax, hydrogenated vegetable oils, mineral oil, polyethylene glycols and the like and mixtures thereof.

Suitable glidants include, but are not limited to, colloidal silica, calcium silicate, magnesium silicate, silicon hydrogel, cornstarch, talc and the like and mixtures thereof.

Sweetening and flavouring agents are essential when the compositions are meant for disintegration in the mouth to mask the taste of drug and to have better feel by the patient.

Fast release compositions of the present invention are not meant for disintegration in the mouth, accordingly compositions of the present invention are free of sweetening agents and flavouring agents.

In yet another embodiment, the present invention provides wet granulation process for preparing pharmaceutical composition comprising entecavir and at least one pharmaceutically acceptable excipient.

Wet granulation process comprise the steps of: (i) sifting and blending one or more excipients including carbonates/bicarbonates of alkali metals or alkaline earth metals optionally with entecavir to form a dry mix, (ii) granulating the dry mix of step no. (i) using drug solution to form granules followed by drying, (iii) blending the granules of step no. (ii) with remaining portion of excipients including acid component, optionally carbonates/bicarbonates of alkali metals or alkaline earth metals and finally compressing in to tablets or filled in to capsules.

When the dosage form is a tablet then it may additionally be coated with an aqueous or non aqueous solution or dispersion of film forming agents.

In another embodiment fast release composition of the present invention comprising entecavir is useful for treating chronic Hepatitis B virus infection.

The invention described herein can further be illustrated by the following examples but these do not limit the scope of the invention.

### EXAMPLE 1-3

### Entecavir Compositions Prepared by Wet Granulation

| | | | |
|---|---|---|---|
| | Example 1 | Example 2 | Example 3 |
| Ingredients | Mg/tablet | Mg/tablet | Mg/tablet |

Intra-granular ingredients

| | | | |
|---|---|---|---|
| Calcium carbonate | 304.2 | 304.2 | 320.2 |
| Pregelatinized starch | 40 | 40 | 40 |
| Sodium starch glycolate | 24 | — | — |
| Croscarmellose sodium | — | 24 | — |
| Alginic acid | — | — | 12 |
| Sodium carboxy methylcellulose | 0.8 | 0.8 | 0.8 |

Drug solution

| | | | |
|---|---|---|---|
| Entecavir | 1 | 1 | 1 |
| Purified water | q.s. | q.s. | q.s. |

Extra-granular ingredients

| | | | |
|---|---|---|---|
| Citric acid monohydrate | 8 | 8 | 4 |
| Alginic acid | — | — | 16 |
| Croscaramellose sodium | — | 16 | — |
| Sodium starch glycolate | 16 | — | — |

Lubrication

| | | | |
|---|---|---|---|
| Sodium stearyl fumarate | 6 | 6 | 6 |
| Total tablet Weight | 400 | 400 | 400 |

### Brief Manufacturing Process:

i) Intra-granular ingredients were sifted and blended together,

ii) entecavir was added to hot water at 60° C. to 70° C. under stirring to get clear drug solution followed by cooling,

iii) the blended material of step no. (i) was granulated using drug solution of step no. (ii) and the resulted granules were dried and milled using a multimill or cone mill,

iv) milled granules of step no. (iii) were sifted through # 30 mesh completely,

v) extra granular ingredients were sifted together through # 40 mesh,

vi) sodium stearyl fumarate was sifted through # 60 mesh,

vii) materials of step no. (iv), (v) and (vi) were blended together and compressed into tablets or filled into capsules,

viii) compressed tablets were optionally coated with Opadry II Pink.

### Study on Dissolution:

Dissolution test was performed for tablets of Example 1 to 3, in 1000 ml of 50 mM phosphate buffer pH 6.8 using paddle method at 50 rpm.

TABLE 1

| | Cumulative % drug release | | |
|---|---|---|---|
| Time in minutes | Example 1 | Example 2 | Example 3 |
| 5 | 96 | 90 | 84 |
| 10 | 97 | 94 | 91 |
| 15 | 98 | 96 | 92 |
| 30 | 99 | 97 | 96 |
| 45 | 99 | 98 | 98 |
| 60 | 100 | 98 | 99 |

### EXAMPLE 4-5

### Entecavir Compositions Prepared by Wet Granulation

| | | |
|---|---|---|
| | Example 4 | Example 5 |
| Ingredients | Mg/tablet | Mg/tablet |

Intra-granular ingredients

| | | |
|---|---|---|
| Calcium carbonate | — | 292.2 |
| Magnesium carbonate | 200.2 | — |
| Pregelatinized starch | 132 | 40 |
| Soy polysaccharide | 32 | 32 |
| Sodium carboxy methylcellulose | 0.8 | 0.8 |

Drug solution

| | | |
|---|---|---|
| Entecavir | 1 | 1 |
| Purified water | q.s. | q.s. |

Extra-granular ingredients

| | | |
|---|---|---|
| Citric acid monohydrate | 8 | — |
| Ascorbic acid | — | 8 |
| Soy polysaccharide | 20 | 20 |

Lubrication

| | | |
|---|---|---|
| Sodium stearyl fumarate | 6 | 6 |
| Total tablet weight | 400 | 400 |

Manufacturing Process: Same as given for Example 1.

### Study on Dissolution:

Dissolution test was performed for tablets of Example 4 to 5, in 1000 ml of 50 mM phosphate buffer pH 6.8 using paddle method at 50 rpm.

TABLE 2

Cumulative % drug release

| Time in minutes | Example 4 | Example 5 |
|---|---|---|
| 5 | 89 | 92 |
| 10 | 94 | 93 |
| 15 | 95 | 94 |
| 30 | 96 | 95 |
| 45 | 97 | 95 |
| 60 | 97 | 96 |

### EXAMPLE-6

### Entecavir Compositions Prepared by Wet Granulation

| | |
|---|---|
| Ingredients | Mg/tablet |

Intra-granular ingredients

| | |
|---|---|
| Calcium carbonate | 292.2 |
| Pregelatinized starch | 40 |
| Soy•polysaccharide | 32 |
| Sodium carboxy methylcellulose | 0.8 |

Drug solution

| | |
|---|---|
| Entecavir monohydrate | 1 |
| Purified water | q.s. |

Extra-granular ingredients

| | |
|---|---|
| Citric acid monohydrate | 8 |
| Soy•polysaccharide | 20 |

Lubrication

| | |
|---|---|
| Sodium stearyl fumarate | 6 |
| Total tablet weight | 400 |

Manufacturing Process: Same as given for Example 1.

### EXAMPLE-7

### Comparative Composition

### Entecavir Compositions Prepared by Dry Granulation Process

| | |
|---|---|
| Ingredients | Mg/tablet |

Intra-granular ingredients

| | |
|---|---|
| Entecavir | 1 |
| Calcium carbonate | 296.2 |
| Soy•polysaccharide | 30 |
| Sodium carboxy methylcellulose | 0.8 |
| Pregelatinized starch | 40 |
| Sodium stearyl fumarate | 2 |

Extra-granular ingredients

| | |
|---|---|
| Soy•polysaccharide | 20 |
| Citric acid | 8 |
| Sodium stearyl fumarate | 2 |
| Total tablet weight | 400 |

### Brief Manufacturing Process:

i) Intra-granular ingredients were sifted and blended together,

ii) the blended material of step no. (i) was slugged/compacted and the resulted slugs/compacts were milled using multimill or cone mill,

iii) milled granules of step no. (ii) were sifted through # 30 mesh completely,

iv) extra-granular ingredients were sifted together through # 40 mesh,

v) sodium stearyl fumarate was sifted through # 60 mesh,

vi) materials of step no. (iii), (iv) and (v) were blended together and compressed into tablets or filled in to capsules,

vii) compressed tablets were optionally coated with Opadry II Pink.

### Comparative Study on Dissolution and Disintegration Time:

Dissolution Profile (in 1000 ml of 50 mM phosphate buffer pH 6.8 using paddle method at 50 rpm) and disintegration time of Baraclude®, Example-6 (composition of the present invention) and Example-7 (composition prepared by dry granulation).

TABLE 3

| Time in | Cumulative % drug release | | | Disintegration time | |
|---|---|---|---|---|---|
| minutes | Baraclude ® | Example 6 | Example 7 | Example 6 | Example 7 |
| 5 | 88 | 92 | 78 | 28 sec | 1-2 min |
| 10 | 93 | 95 | 82 | | |
| 15 | 95 | 97 | 85 | | |
| 30 | 97 | 98 | 90 | | |
| 45 | 98 | 99 | 93 | | |
| 60 | 98 | 99 | 96 | | |

Comparison of blend uniformity for Example 6 and 7:

TABLE 4

% labeled amount

| S. No. | Example 6 | Example 7 |
|---|---|---|
| 1 | 104 | 95 |
| 2 | 102 | 103 |
| 3 | 103 | 102 |
| 4 | 104 | 106 |
| 5 | 103 | 107 |
| 6 | 105 | 106 |
| 7 | 104 | 102 |
| 8 | 104 | 102 |
| 9 | 102 | 102 |
| 10 | 105 | 101 |
| RSD (%) | 1.06 | 3.31 |

The pharmaceutical composition prepared in Example 6 (wet granulation) and 7 (dry granulation) were tested for dissolution, disintegration and blend uniformity.

Results from Table 3, reveals that entecavir compositions of the present invention prepared by wet granulation have better dissolution and disintegration time.

The final blend was sampled with ten samples taken from different places in the storage container, and every sample was tested for assay. The results are summarized in Table 4, where “RSD” refers to the relative standard deviation. Thus as illustrated in Table 4, entecavir compositions of the present invention prepared by wet granulation have acceptable RSD limits, while those prepared by dry granulation suffer from a lack of blend uniformity.

### EXAMPLE-8

### Compositions of Entecavir Tablets (Free of Acid Component)

| | |
|---|---|
| Ingredients | Mg/tablet |

Intra-granular ingredients

| | |
|---|---|
| Calcium carbonate | 302.2 |
| Pregelatinized starch | 40 |
| Soy•Polysaccharide | 32 |
| Sodium carboxy methylcellulose | 0.8 |

Drug solution

| | |
|---|---|
| Entecavir | 1 |
| Purified water | q.s. |

Extra-granular ingredients

| | |
|---|---|
| Soy•Polysaccharide | 20 |

Lubrication

| | |
|---|---|
| Sodium stearyl fumarate | 4 |
| Total tablet weight | 400 |

Manufacturing Process: Same as given for Example 1.

### Comparative Study on Dissolution:

Dissolution test was performed for tablets of Example 6 and 8, in 1000 ml of 50 mM phosphate buffer pH 6.8 using paddle method at 50 rpm.

TABLE 5

| | |
|---|---|
| Time in | Cumulative % drug release |

| minutes | Example 8 | Example 6 |
|---|---|---|
| 5 | 78 | 92 |
| 10 | 83 | 95 |
| 15 | 86 | 97 |
| 30 | 90 | 98 |
| 45 | 94 | 99 |
| 60 | 96 | 99 |

Results from Table 5, reveal that % drug release is better and comparable with Baraclude® in example 6 of the present invention (containing acid component) when compared with example 8 (entecavir compositions free of acid component).

1. A fast release pharmaceutical composition comprising entecavir, an acid component, a carbonate or a bicarbonates of an alkaline earth metals, a superdisintegrant, and one or more pharmaceutically acceptable excipients.

2. The pharmaceutical composition according to claim 1, wherein said acid component is citric acid, tartaric acid, fumaric acid, ascorbic acid, or combination thereof.

3. he pharmaceutical composition according to claim 1, wherein said alkaline earth metal is magnesium or calcium.

4. The pharmaceutical composition according to claim 1, wherein said superdisintegrant is sodium starch glycolate, croscarmellose sodium, a soy polysaccharide, cross-linked alginic acid, gellan gum, or xanthan gum.

5. The pharmaceutical composition according to claim 1, wherein said superdisintegrant is a soy polysaccharide.

6. The pharmaceutical composition according to claim 1, wherein said pharmaceutically acceptable excipients is a diluent, a binder, a lubricant, a glidant, or a combination thereof.

7. The pharmaceutical composition according claim 1, wherein the composition is free of sweetening agents and free of flavouring agents.

8. The pharmaceutical composition according to claim 1, wherein the composition is prepared by a wet granulation method.

9. A pharmaceutical composition comprising:
i) 0.05-1% by weight of entecavir,
ii) 1-6% by weight of an acid component,
iii) 1-90% by weight of a carbonates or bicarbonates of magnesium or calcium, and
iv) 1-20% by weight of a soy polysaccharide as a superdisintegrant,
wherein all amounts are based on a total weight of the composition.

10. The pharmaceutical composition of claim 9, wherein the composition is prepared by a wet granulation method.

11. The fast release pharmaceutical tablet composition of claim 1 comprising, citric acid, calcium carbonate and a soy polysaccharide; wherein the composition is prepared by a wet granulation method.

12. A process for preparing compositions of entecavir by wet granulation method comprising:
(i) sifting and blending a carbonate or bicarbonate of an alkali metal or alkaline earth metal and optionally the entecavir to form a dry mix,
(ii) granulating the dry mix of step no. (i) using a solution containing the entecavir to form granules, followed by drying to produce dry granules,
(iii) blending the dry granules of step no. (ii) with one or more additional excipients to from a blend, and compressing the blend in-to tablets or filling the blend in to capsules.

13. The process according to claim 12, wherein the composition comprises a superdisintegrant.

14. The pharmaceutical composition according to claim 1, wherein the composition is a composition for oral administration in the form of tablets, capsules, granules, mini-tablets or and fast disintegrating tablets.

15. (canceled)

16. The pharmaceutical composition according to claim 9, wherein the composition is a composition for oral administration in the form of tablets, capsules, granules, mini-tablets, or fast disintegrating tablets.

17. The pharmaceutical composition according to claim 11, wherein the composition is a composition for oral administration in the form of tablets, capsules, granules, mini-tablets, or fast disintegrating tablets.
